(19) United States
(12) Patent Application Publication
(10) Pub. No.: US 2011/0001926 A1
(43) Pub. Date: Jan. 6, 2011

(54) Device and Method for Performing Measurements During a Surgical Intervention by Means of an Optical Coherence Tomography Device

(75) Inventors: Dieter Mann, Mainaschaff (DE); Hans Hoerauf, Sierksdorf (DE); Christian Winter, Lubeck (DE); Ralf Engelhardt, Lubeck (DE)

Correspondence Address: MCDONNELL BOEHNEN HULBERT & BERGHOFF LLP, 300 S. WACKER DRIVE, 32ND FLOOR, CHICAGO, IL, 60606, US

(73) Assignees: DIETER MANN GMBH, Mainaschaff (DE); HEIDELBERG ENGINEERING GMBH, Heidelberg (DE)

(21) Appl. No.: 12/446,433

(22) PCT Filed: Sep. 19, 2007
(86) PCT No.: PCT/EP07/08169

§ 371 Date: Sep. 21, 2010

(30) Foreign Application Priority Data
Sep. 19, 2006 (DE) 10 2006 043 889.2

(51) Int. Cl. G02B 6/32; A61B 3/10

(52) U.S. Cl. 351/205; 385/33

(57) ABSTRACT

A device for a therapeutic treatment of the eye by means of a laser is provided, which allows a real-time monitoring of the treatment. In particular, the laser light is supplied to the treatment region via a fibre. The monitoring of the treatment happens by means of optical coherence tomography (OCT). To this end the OCT measurement beam and the treatment laser light are coupled in a probe that is put onto the eye and allows to focus the OCT measurement beam on the tissue region inside of the eye that is treated at that moment.

The present invention is related to a device and a method for performing measurements during a laser-surgical intervention by means of optical coherence tomography (OCT).

OCT is a method sufficiently described in the literature, which is based on the physical principle of white light interferometry. The different technical embodiments are not uniformly termed in the literature (LCOT, TD-OCT, etc.).

Optical coherence tomography is an examination method wherein temporally incoherent light is applied for distance measurements by using an interferometer. For instance light that is generated by a LED is splitted into two portions by means of a beam splitter. One portion is reflected at a reference mirror, the other portion is reflected at the tissue to be examined. The interference of the reflected light rays takes place in a detector. From the resulting pattern it is possible to determine the relative optical path length of the light from the tissue with respect to a reference light. Thus it is possible to obtain an information about the depth dependence of the backscatter in the tissue to be examined.

Due to the provision of a depth information point by point and due to the non-contact measurement the examination method is particularly suitable for an examination of the eye, mainly the fundus of the eye, but also is suitable for an examination of the anterior eye sections.

Trans-scleral cyclophotocoagulation (TSCPC) is a method that is applied in patients, in which a lowering of the intra-ocular pressure in a different way (e.g. by medication) is not successful. Specifically, the ciliary body is damaged by a laser through the sclera, whereby the ability of the ciliary body to release water into the posterior eye chamber is reduced and the intra-ocular pressure, which in the long run is dangerous for the optic nerve, is lowered. Though at first laser radiation was applied by using a slit lamp, nowadays a (contact) method has established itself, wherein the radiation is applied by means of a fibre optics using a specific probe, which is put directly onto the eye. The reason for deviating from a slit lamp arrangement are improved aiming and focussing capabilities when using the contact method. Moreover, the use of the contact method results in an improved transmission of light in the eye, whereby the energy of the laser can be deposited in a better way on the other side of the sclera and a reduced damage of the sclera occurs. The reason for this is that the transmission of the sclera is remarkably improved when the probe is put onto the eye due to the pressure that is exerted on the sclera (see with respect to this for example Vogel et al., Lasers Surg Med 1991; 11:331-340).

The disadvantage of the trans-scleral cyclophotocoagulation when using the contact method is that up to now it is not possible to monitor in real time the damage to the ciliary tissue by the laser. However, an overdosage can lead to unintentional vaporization of tissue (so-called “pop effect”), an intensified inflammatory reaction and further complications. On the other hand an underdosage of the applied laser power has no therapeutic effect. To make matters worse the coagulation effects in the ciliary body vary very much from patient to patient. This can for example be due to different absorption (degree of pigmentation) or a different position of the ciliary body because of a different thickness of the layers above it. Furthermore, the achieved lowering of the intra-ocular pressure depends on the type of glaucoma, the age of the patient and further factors.

The European Patent EP 1 231 496 B1 discloses a surgical device, which is controlled by optical coherence tomography. There, the amount of tissue modification during a laser treatment is monitored and controlled by an OCT device. The equipment, however, makes use of an opthalmological surgery microscope, in which the laser beam that is used for the surgical treatment is guided in air by means of a lens system. An application of such a system to a surgical laser, which is applied by means of an optical fibre, is not possible.

Therefore, the object of the present invention is to provide a device and a method, by which laser-surgical procedures may be monitored in real-time, when the laser, which is used for a therapeutic treatment, applies the light power to the tissue to be treated by means of a fibre.

The object is achieved by a coupling element according to claim 1, a device according to claim 6 and a method according to claim 12. Further developments of the invention are described in the dependent claims.

Further features and the further usefulness of the present invention will arise from the description of embodiments relating to the attached drawings, of which:

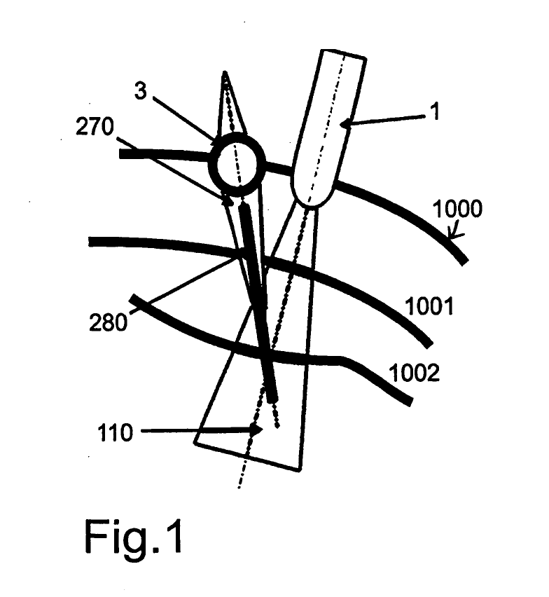

FIG. 1 shows a schematic representation of the method according to the invention,

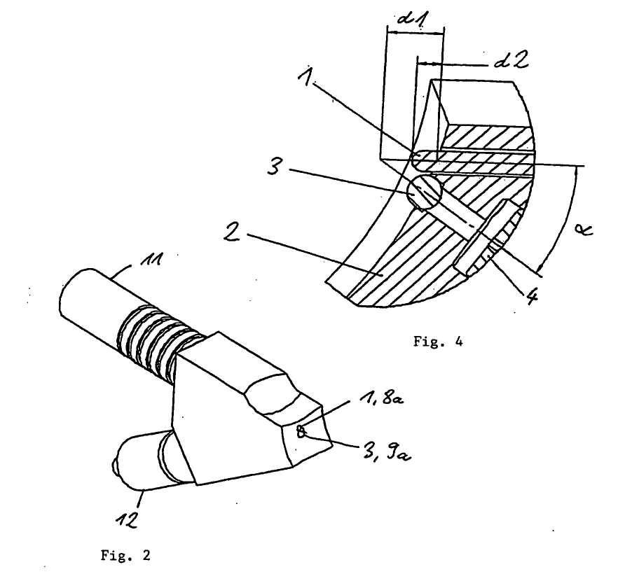

FIG. 2 shows a coupling element according to the invention,

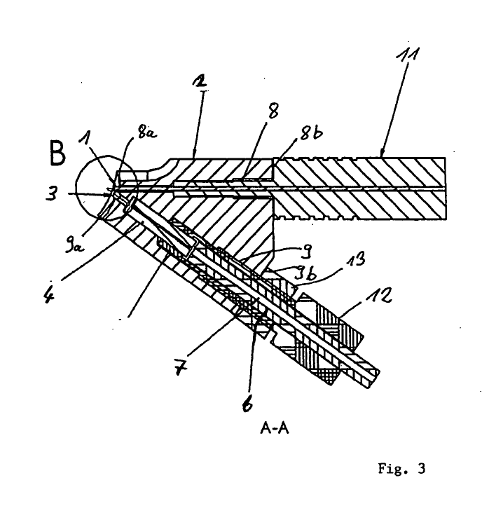

FIG. 3 shows a cross-section of the coupling element that is shown in FIG. 2,

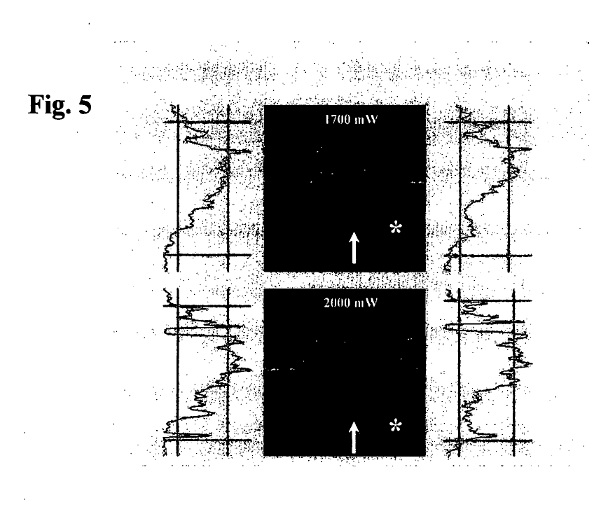

FIG. 4 shows an enlarged representation of the front end region of the coupling element, which is designated by B in FIG. 3,

FIG. 5 shows the time dependence of an OCT depth profile in the region of the ciliary bodies for three different powers of the treatment laser and

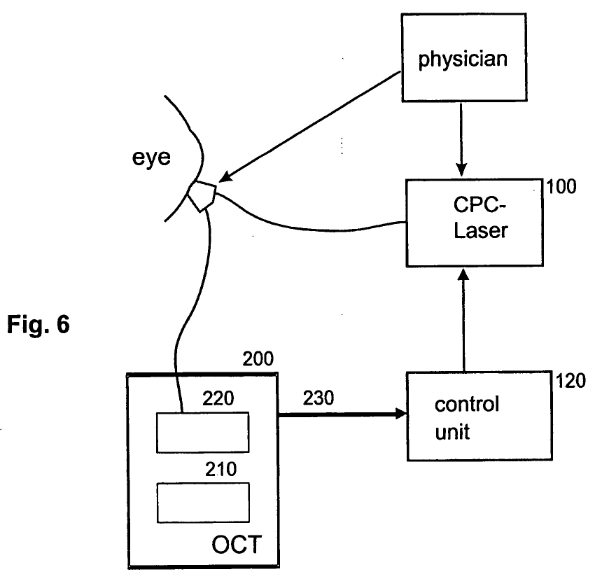

FIG. 6 shows a schematic representation of a real-time-OCT-laser treatment device.

According to the invention the tissue in the treatment region is examined by means of an OCT measurement before, during and after the treatment with the therapeutic laser. In an embodiment in the following a trans-scleral cyclophotocoagulation is described as therapeutic treatment. However, the invention is also applicable to other laser treatments.

The general setup of a system for a real-time OCT laser treatment device is shown schematically in FIG. 6. The OCT device 200 comprises a reference beam unit 210 and a measurement beam unit 220. Though these two are shown separately, of course it is also possible that a single unit fulfils both functions, when e.g. the reference beam is generated in the optical path of the measurement beam by means of a semi-transparent mirror or a reflection in the optical path is used as reference. In order to examine in real-time, the conditions at the coagulation spot it is proposed to focus the measurement beam of the OCT device 200 on the optical path of the laser 100 for performing the cyclophotocoagulation. This is schematically shown in FIG. 1. In the Figure reference number 1 designates a first optical fibre of the CPC laser, which is put onto the eye 1000 near the corneal limbus. The optical path of the CPC laser that penetrates the outer edge of the sclera, which is designated by 1001, and the outer edge of the ciliary body, which is designated by 1002, is designated by the reference number 110. As can be seen in the Figure, near the position, where the CPC fibre 1 is put onto the eye, a spherical lens 3 is put onto the eye, which serves to focus an OCT beam 270 from an OCT device 200 (not shown). As can be derived from the Figure, the focus lies inside of the ciliary body in the region of the CPC beam 110. The measurement region 280 of the OCT device 200, in which a depth information is obtained via the backscattering of the tissue, is also schematically shown in the Figure.

The schematic setup that is shown in FIG. 1 can be implemented by means of a coupling element, which is represented in FIGS. 2 to 4. As can best be seen in the sectional representation of FIG. 3, the coupling element comprises a connecting element 2, which in a section has roughly the shape of a triangle. In the connecting element 2 two channel-like reception sections 8 and 9 are provided, each of which has a front end 8a, 9a and a back end 8b, 9b. An optical fibre 1 has been inserted into the first channel-like reception section 8 from the back end 8b. A second optical fibre 7 has been inserted into the channel-like reception section from the back end 9b. As can be seen, the diameter of the channel-like reception sections is larger than the one of the optical fibres 1, 7, so that the optical fibres in the reception sections can be partially surrounded by a ferrule (guiding tube) 4 or a plastic cover 6. Furthermore, it can be seen that both channel-like reception sections 8 and 9 are close to each other with its front ends 8a, 9a. The distance of the channel centres there is roughly one millimetre. Moreover, both channel-like reception sections include an angle α. In the present example the fixing of the optical fibres to the connecting element 2 is done by means of screws, wherein in each case the fibres are mounted in a clamping sleeve 11 or 12 together with a cover 6 or a ferrule 4. The clamping sleeves are screwed into the connecting element 2, wherein it is possible to interlock them by tightening a nut (13). An adjustment of the position of the optical fibres in the reception sections is done by twisting the clamping sleeves 11, 12. In the example, which is shown, the length of the reception sections is approximately 3 cm and the ferrule 4 that is shown for the optical fibre 7 has a length of approximately 1 cm.

By the just described mounting of the optical fibres 1, 7 in the reception sections 11, 12 a crossing of the optical axes of both optical fibres at a defined distance from the front ends 8a, 9a of the reception sections is achieved. The lens element 3 (for example a spherical lens or ball lens), which is shown in FIG. 1, is mounted at the front end 9a of the second reception section 9 in such a way that light, which is leaving the optical fibre 7, is focussed on the optical axis of the light, which is leaving the optical fibre 1. The cross is at a distance d1 of approximately 1.6 mm from the front ends 8a, 9a.

When performing a trans-scleral cyclophotocoagulation, the treatment laser beam is applied via the optical fibre 1 and the shown coupling element is put onto the eye with its side, on which the front ends 8a, 9a of the reception sections are located. For this purpose the connecting element 2 has a concave shape at the contact surface, which is applied upon the eye. The measurement beam of the OCT device 200 is applied via the second optical fibre 7.

The size of the angle α, which is included by both channel-like reception sections, correlates with the desired distance of the cross of the light rays, which are emitted by the fibres 1 and 7, from the front ends, which distance depends on the type of therapeutic laser treatment. Furthermore, there is a dependence on the distance of the front ends 8a, 9a from each other.

In a CPC the treatment region (that is to say the ciliary body) is inside of the eye approximately 1.6 mm away from the contact surface between the coupling element and the eye. Accordingly, in the embodiment that is shown here, a value of 35° was chosen for the angle α.

When the invention is put into practice, one should aim at making the angle α as small as possible, so that the OCT beam can enter into the eye almost at a right angle, when the treatment beam enters the eye perpendicularly to the contact surface on the eye. This can be achieved by choosing the lens element 3 to be as small as possible, so that the distance of the front ends 8a, 9a is as small as possible.

In the OCT device 200 preferably a light source having a wavelength λ of 1310 nm is used (for example an infra-red super luminescence diode SLD-561 of the company Super LUM, Moscow, Russian Federation, having a coherence length of 20 μm and a luminous power of approximately 500 μW). As treatment laser for example an infra-red laser diode of IRIDEX Corporation, Mountain View, U.S.A. (e.g. IRIS Medical Oculight SLx) can be used, which has a wavelength of 810 nm and a laser power of 1.5 to 2.5 W. However, the invention is not limited to the previously mentioned light sources.

The larger the wavelength the larger the transmission of light through the sclera. Out of this reason for the OCT measurement preferably a light source having a longer wavelength is used. As a transition to a longer wavelength is not offhand possible for the laser light of the treatment laser, it is advantageous to increase the transmission by additional means. As mentioned above, a possible approach is to apply pressure onto the sclera leading to an increase of the transmission through the sclera. The CPC fibre 1 protrudes from the concave surface of the connecting element 2 by a value d2 of approximately 0.75 mm, so that it is possible to apply pressure. Furthermore, the fibre is spherically rounded in order to avoid injuries.

The concave region of the connecting element 2 is shown at a larger scale in FIG. 4. As can be seen, the ball lens 3 also protrudes from the concave face. Thereby it is possible to create a pressure channel in the sclera at the same time for the OCT beam and the CPC laser beam. This improves the quality of the OCT measurement and makes it possible to enter the wavelength region around 800 nm also with the OCT measurement beam.

In the OCT device that was used the optical retardation in the reference arm was approximately 2.5 mm in air. When taking into account a different refractive index, from this a depth region for the OCT measurement of approximately 1.8 mm at an axial resolution of approximately 15 μm results depending on the optical properties of the tissue that is examined.

With the device that was just described, CPC treatments with a simultaneous OCT monitoring were performed at four patients that did not respond to other glaucoma treatments. The signal/noise ratio was approximately 95 dB.

FIG. 5 shows the variation over time of the OCT depth profile at a position for two different powers of the treatment laser. The measurement was made at 1310 nm, 100 Hz and an axial depth of 1.8 mm. In the pictures the tissue that was treated is at the level of the white asterisk, which can be recognized from the fact that at the start of the treatment there is little backscattering (dark). Only after a point in time that is marked by a white arrow, at which the treatment laser was switched on, the backscattering changes in this region, wherein the change is more remarkable for 2000 mW than for 1700 mW.

In order to show the results in a clearer way, in FIG. 5 at the left side and at the right side of the time scans the averaged intensity distributions between 0 s and 0.33 s and between 1.67 s and 2 s are plotted. Also a comparison of the intensity distributions at the start and at the end of the measurement over time clearly shows the change of the treated tissue.

Therefore, it can be seen that the device according to the invention makes possible a real-time monitoring of a CPC treatment.

By the real-time monitoring it is possible to control the laser power of the treatment laser in dependence of the result of the OCT recordings. As is shown in FIG. 6, to this end an output signal 230, which was derived from the depth profile of the OCT device, is supplied to a control element 120 of the treatment laser 100. This output signal 230 raises or lowers the power of the treatment laser 100 in dependence of this signal 230. In particular it is possible that the control element 120 is configured in such a way that it switches off the laser 100 in dependence of a certain output signal 230 of the OCT device in order to avoid an unintentional damage of the tissue.

It shall also be noted that the invention is not limited to a particular embodiment of the OCT, but may be implemented with all OCT devices that are known from the literature.

In a variation of the embodiment a known probe for the trans-scleral cyclophotocoagulation in a contact method is used, wherein the OCT beam and the treatment beam are supplied in such a way that they are already superimposed on one another when entering the probe. Thereby it can be automatically accomplished that the OCT beam enters the eye as perpendicular as possible.

1. Coupling element for light having
a connecting element (2) comprising
a first channel-like reception section (8) having a front end (8a) and a back end (8b) and
a second channel-like reception section (9) having a front end (9a) and a back end (9b),
a first optical fibre (1) that is inserted into the first channel-like reception section (8) from the back end (8b) such that light can be emitted at its front end (8a) and
a second optical fibre (7), which is inserted into the second channel-like reception section (9) from the back end (9b) such that light can be emitted at its front end (9a), wherein
the first and the second channel-like reception sections (8, 9) include an angle (α) and
the second channel-like reception section (9) has a lens element (3) at its front end (9a), which is mounted in such a way that it focuses light, which is supplied via the second optical fibre (7), on the beam path of light, which is emitted at the front end (8a) of the first channel-like reception section (8).

2. Coupling element according to claim 1, wherein the outer surface of the connecting part (2) has a concave shape at the joining of the front ends (8a, 9a) of the first and second channel-like reception sections (8, 9), so that the coupling element can be put onto an eye with the front ends.

3. Coupling element according to claim 2, wherein the first optical fibre (1) is mounted in the first channel-like reception section (8) such that it protrudes between 0.5 mm and 1 mm from the concave contact surface that is put onto the eye.

4. Coupling element according to claim 2 or 3, wherein the focussing element (3) protrudes between 0.5 mm and 1 mm from the concave contact surface that is put onto the eye.

5. Coupling element according to one of claims 1 to 4, wherein the angle (α) which is included by the first and the second channel-like reception sections (8, 9), is smaller than 35°.

6. Device for a therapeutic treatment of the eye by means of a laser having
a laser (100),
an optical fibre (1) for supplying the laser light to the tissue to be treated,
an optical coherence tomography (OCT) device (200) for determining a depth-resolved backscattering property of the tissue to be examined by means of a measurement beam and
a coupling element to be put onto the eye, wherein the treatment beam of the laser (100) and the measurement beam of the OCT device (200) are brought together in such a way that during the therapeutic treatment of the tissue by means of the laser (100) an examination of the treated tissue by means of the OCT device (200) is possible.

7. Device according to claim 6, wherein the treatment beam of the laser (100) and the measurement beam of the OCT device (200) are supplied to the coupling element in an already super imposed condition.

8. Device according to claim 6, wherein the coupling element is a coupling element according to one of claims 1 to 5.

9. Device according to one of claims 6 to 8, which is suited for performing a trans-scleral cyclophotocoagulation.

10. Device according to one of claims 6 to 9 that further comprises a control element (120) that is adapted to adjust the power of the laser (100) based on an output (230) of the OCT device (200).

11. Device according to claim 10, that is suitable to switch off the laser (100) based on an output (230) of the OCT device (200).

12. Method for examining tissue structures in the eye by means of optical coherence tomography, wherein
the measurement beam of an optical coherence tomography (OCT) device (200) is supplied to the examined object by means of an optical fibre (7) and
the measurement beam is focused on the optical path of the laser (100) of a device for performing a contact cyclophotocoagulation (CPC), so that simultaneously to the cyclophotocoagulation procedure a depth-resolved backscattering of the tissue in the treatment region can be measured.

13. Method according to claim 12, wherein a variation over time of the backscattering of the tissue is recorded and output.

14. Method according to claim 12 or 13, wherein a device according to one of claims 1 to 11 is used.
